(19) United States
(12) Patent Application Publication
(10) Pub. No.: US 2011/0270197 A1
(43) Pub. Date: Nov. 3, 2011

(54) Ejection Device

(75) Inventors: David Weill, Begnins (CH); Pierre-Yves Chassot, Thoiry (FR)

(73) Assignee: Primequal SA, Geneva (CH)

(21) Appl. No.: 13/143,947

(22) PCT Filed: Jan. 14, 2010
(86) PCT No.: PCT/EP2010/050411

§ 371 Date: Jul. 11, 2011

(30) Foreign Application Priority Data
Jan. 16, 2009 (FR) 0900199

(51) Int. Cl. A61M 5/31

(52) U.S. Cl. 604/187

(57) ABSTRACT

Device for ejecting a liquid or pasty product, comprising a body (2) having a housing for holding a cartridge (10) intended to contain the liquid or pasty product to be ejected, characterized in that the body (2) comprises a means for introducing the cartridge (10) into its housing in a relative direction with respect to the housing, said direction being different from a longitudinal direction by way of the rear.

The invention relates to a device for ejecting a liquid or pasty product. It also relates to a method of putting together or manufacturing such a device for ejecting a liquid or pasty product, and also a kit for putting together such a device.

A conventional and very simple device for ejecting a product, which is used in particular in the medical or dental fields, is in the form of an approximately cylindrical rectilinear syringe comprising a front cartridge containing the product and a cylindrical and rectilinear rear plunger that is able to move translationally within the cartridge. An operator simply pushes the plunger, which slides inside the cartridge, ejecting the product through the front end of the cartridge, on which there is mounted a needle. The production of such a syringe meets stringent hygiene and sterility requirements, in particular as far as the integration of the product into the cartridge is concerned. A drawback with this solution is that it is difficult to handle such a syringe because pushing the plunger is not very user-friendly, and it is almost impossible to control the force required to eject a precise dose at a controlled speed.

Patent Application FR2913341 discloses a different ejecting device, which comprises a body comprising a front container-holder part and a rear part, it being very easily possible to put these two parts together. In addition, a lever incorporating a pawl is mounted such that it can move on the body and acts on a rack, which moves translationally in the central part of the body in order to act on a plunger that enables the ejection of the product contained in the container holder. This solution makes it possible to control the quantity of product ejected, since each manipulation of the lever causes the rack to move forwards progressively, tooth by tooth. In addition, each movement forwards produces an audible click by contact with a non-return pawl, thereby making it possible to easily control aurally the quantity of product ejected. The use of a flexible, elastic and user-friendly lever additionally makes it possible to operate with less force, thereby increasing the control of the quantities and speeds of product ejected. However, one difficulty with such a solution consists in filling the container holder with product to be ejected by a method that is compatible with the constraints of hygiene and/or sterility in the medical or dental field or is linked to the method of manufacturing the contained product. For this purpose, it is envisaged to place into the container holder a separate cartridge containing the product to be ejected, before the main body is finally put together. A drawback with such a solution resides in the fact that it is necessary to manufacture a specific cartridge that is specially adapted to this device and will be housed in the container holder of the device by being introduced through the rear of the container holder before the rear part of the body of the device is fitted to the container holder. This drawback leads to a high cost of packaging the product in a cartridge in the medical field.

Thus, a general object of the invention is to provide a solution for ejecting a liquid or pasty product which does not comprise any or some of the drawbacks associated with existing solutions.

A first object is to provide a solution for ejecting a liquid or pasty product that affords a maximum degree of hygiene.

A second object is to provide a solution for ejecting a liquid or pasty product that is simple and cost-effective.

A third object is to provide a user-friendly solution for ejecting a liquid or pasty product that makes it possible to control with precision the quantity of product ejected.

To this end, the invention is based on a device for ejecting a liquid or pasty product, comprising a body having a housing for holding a cartridge intended to contain the liquid or pasty product to be ejected, characterized in that the body comprises a means for introducing the cartridge into its housing in a relative direction with respect to the housing, said direction being different from a longitudinal direction by way of the rear.

The means for introducing the cartridge into its housing may make it possible to introduce the cartridge in a relative direction with respect to the housing, said direction comprising a non-zero component in a plane perpendicular to the longitudinal axis, or the means for introducing the cartridge into its housing may make it possible to introduce the cartridge in a relative direction with respect to the housing, said direction being approximately perpendicular to the housing.

The means for introducing the cartridge into its housing may consist of a body of the ejecting device that comprises at least two separate parts that are put together around the cartridge.

For this purpose, the body of the ejecting device may comprise a front container-holder part which is composed of two half shells and a rear part.

The means for introducing the cartridge into its housing may consist of a body of the ejecting device comprising a slot that extends in the longitudinal direction.

The slot may extend over a length that represents at least one third of the length of a front container-holder part of the body of the ejecting device.

The body of the ejecting device may comprise mechanical means for fastening the cartridge, such as clips.

The body may comprise in its rear part a rack that moves translationally and is able to act on a plunger positioned at the rear end of a cartridge intended to contain the product, and a hinged lever and a pawl, for acting on the rack, that form a monolithic structure made of plastic, the lever comprising a cylindrical rear end that forms its rotation pivot.

The device for ejecting a liquid or pasty product may comprise a cartridge arranged in a housing of the body of the device, this cartridge comprising at least one tool holder arranged at least partially in front of the front face of the body of the ejecting device.

The cartridge may comprise a locking device of the Luer lock type for locking the tool to the tool holder, this locking device having a part which has a diameter greater than that of the cartridge arranged in front of the body of the ejecting device, and the front face of the ejecting device may comprise a circular opening delimited by a ring which has a diameter that corresponds to that of the cartridge.

The locking device may have a groove in which a part of the body of the ejecting device is arranged in order to hold this locking device.

The ejecting device may comprise a rack that can move translationally and is fixed to a plunger positioned within the cartridge by a screw-type means.

The invention also relates to a kit for putting together an ejecting device as defined above, comprising the following non-assembled separate elements: one or more part(s) of a body comprising a housing and a means for introducing a cartridge into this body, a lever, a rack, and a cartridge comprising a liquid or pasty product to be ejected.

The invention also relates to a method of putting together a device for ejecting a liquid or pasty product as defined above, characterized in that it comprises a step that consists in introducing a cartridge into the body of the ejecting device in any direction other than longitudinal introduction by way of the rear.

The method of putting together an ejecting device may comprise a step of putting together at least two separate parts of the body of the device around a cartridge.

Finally, the method may comprise a step of introducing a cartridge by way of a longitudinal slot in the body of the ejecting device.

These subjects, features and advantages, and others of the present invention, will be explained in detail in the following description of particular non-limiting embodiments in conjunction with the attached figures, in which:

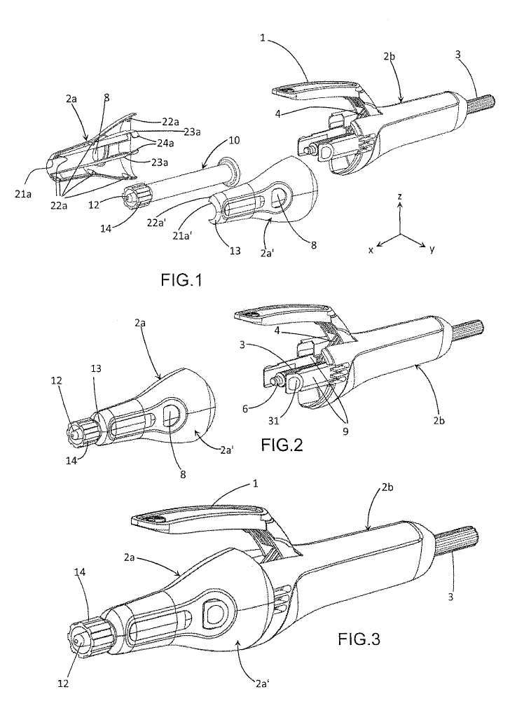

FIG. 1 shows a partially exploded perspective view of an ejecting device according to a first embodiment of the invention,

FIG. 2 shows an intermediate stage in the putting together of the ejecting device according to the first embodiment of the invention,

FIG. 3 shows a perspective view of the ejecting device according to the first embodiment of the invention,

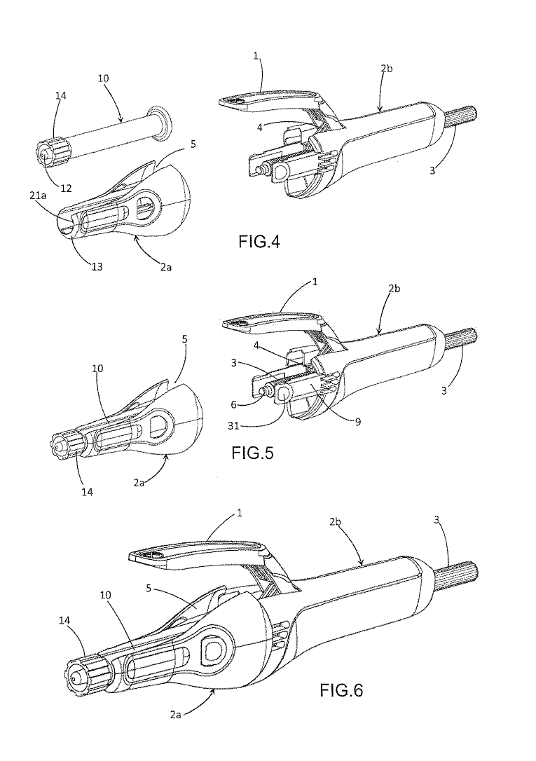

FIG. 4 shows a partially exploded perspective view of an ejecting device according to a second embodiment of the invention,

FIG. 5 shows an intermediate stage in the mounting of the ejecting device according to the second embodiment of the invention,

FIG. 6 shows a perspective view of the ejecting device according to the second embodiment of the invention,

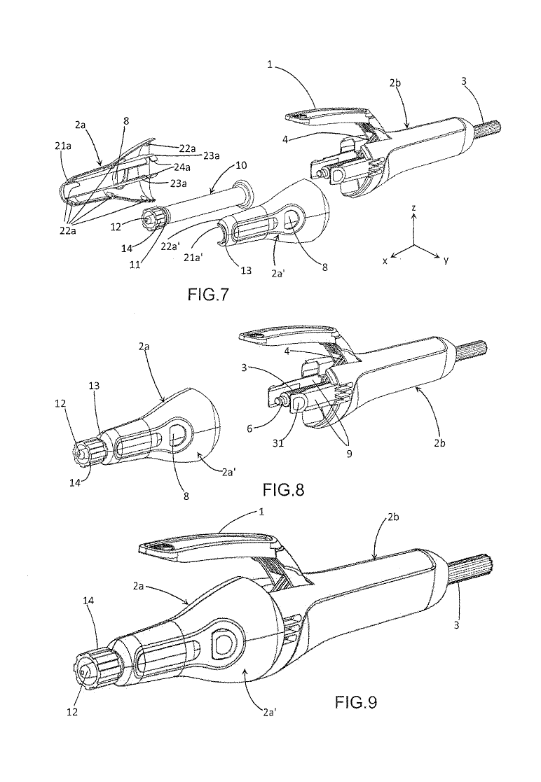

FIG. 7 shows a partially exploded perspective view of a variant of the first embodiment of the invention,

FIG. 8 shows an intermediate stage in the mounting of the ejecting device according to the variant of the first embodiment of the invention,

FIG. 9 shows a perspective view of the ejecting device according to the variant of the first embodiment of the invention,

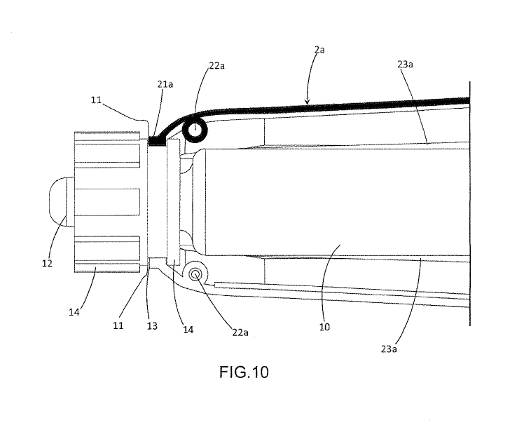

FIG. 10 shows a side view in a transverse direction of the front part of the ejecting device according to the variant of the first embodiment of the invention, in which one half shell has been removed.

For the following description, the longitudinal axis extending from the rear of the ejecting device to its front part will be called x, the vertical axis perpendicular to the axis x and passing through the middle part of the lever will be called z, and the transverse axis perpendicular to the two previous axes x, z will be called y, as is shown in the figures.

The description of the embodiments will be given in the context of an ejecting device serving as a syringe intended for the medial field for injecting a product into the gums. It will be specified below that the invention can be used in other fields. In addition, for the description of the various embodiments, the same references will be used for the same components.

The concept of the invention consists in combining the use of a cylindrical cartridge of a standard rectilinear syringe, comprising a product to be ejected that is packaged according to a long-known method and that meets the constraints of hygiene and/or sterility and/or the manufacturing constraints, with an ejecting device that comprises a user-friendly and well-performing ejection mechanism that operates, for example, according to the principle of the device described in document FR2913341. For this purpose, the solution is based on the introduction of the cartridge, which we shall also call more simply a syringe, into a housing formed in the body of the ejecting device. Such a cartridge or syringe consists of a main cylindrical reservoir made of plastic or glass, on the front part of which there is arranged a tool holder intended for the fastening of a needle and in the rear part of which there is housed a plunger which is intended, on account of its ability to move translationally, to eject the product contained in the reservoir.

Thus, the ejecting device of the present invention has the advantage of being compatible with conventional methods of packaging the medical product within a syringe cartridge, without it being necessary to modify these methods or the components used, while benefiting from the advantages of controlled ejection of the product by way of the mechanics offered by the ejecting device.

FIG. 1 shows an exploded perspective view of one embodiment of the invention. The ejecting device consists of a main body obtained by putting together three separate components produced by injection moulding of plastic. This body comprises two front parts 2a and 2a′ in the form of half shells which are intended to be connected around the cartridge 10 in order to form a front part, known as a “container holder”, of the body of the device, and a rear part 2b that incorporates the mechanism for ejecting the product, as will be explained hereinbelow.

In its front part, the cartridge 10 comprises a tool holder 12 for holding a needle, which is not shown, and a locking device 14, of the type known and standardized under the name “Luer lock”, which is in the form of a ring fixed around the body of the cartridge or integrated into the cartridge so as to prevent the needle from detaching. Specifically, the high pressures exerted on the product in order to eject it are transmitted to the front part of the cartridge and the tool holder 12 risks being ejected forwards from the cartridge 10 at high speed, in the manner of a dart. Such a situation is of course very dangerous for the patient. The function of the Luer lock 14 is to reduce this risk. However, it has a greater diameter than the diameter of the rest of the cartridge, since it is fixed on its outer periphery, thereby complicating the introduction of such a cartridge into a housing in the body of the ejecting device. It cannot, for example, be introduced into the housing in a longitudinal direction from the rear of the container holder since the locking means could not pass through the circular opening provided on the front face 13 of the device, the diameter of which corresponds to that of the cartridge in order to hold the latter in position.

The two front half shells 2a and 2a′ of the container holder, which are approximately symmetrical with respect to a vertical longitudinal mid-plane xz of the ejecting device, comprise different clips 22a and 22a′ that engage with one another in order to put together the two half shells and thus form the container holder. This assembly is obtained by moving the two half shells together in a direction approximately perpendicular to the longitudinal axis of the cartridge.

On the other hand, at least one of the two front parts 2a, 2a′ has clips 23a that are intended to be clipped to the circumference of the cartridge 10 in order to position it properly and to hold it in its housing that is formed within the container holder. For this purpose, two clips 23a, which have a section approximately in the form of a circular arc, can cover an upper and lower surface, respectively, of the circumference of the cartridge. The side ends 24a of the two clips 23a are separated by a distance in the vertical direction z which is less than the diameter of the central cylindrical part of the cartridge 10. During the assembly of the cartridge 10 in the container holder, these two clips 23a move apart by elasticity and then close again around a part of the circumference of the cartridge, thereby holding it in position. Advantageously, these clips extend longitudinally over a large surface area of the cartridge, over at least half the length of the cartridge, in order to ensure that it is held in position in a well-performing manner. Alternatively, any other means of holding the cartridge in position could be envisaged. In addition, the two half shells have two half rings 21a, 21a′ which form a circular opening at the front face 13 of the container holder, the diameter of which opening corresponds to that of the cartridge 10 in order to rest against the main body of the cartridge 10 just behind the Luer lock 14 when the two half shells are put together.

Finally, the ejecting device comprises a rear body 2b, within which a rack 3 is positioned in a central cylindrical bore. On the upper surface of this rear body 2b, a lever 1 comprising a pawl 4 is mounted such that it can move in a hinged manner about an axis, such that the lower end of the pawl 4 engages with the rack 3. By way of this mechanism, the actuation of the lever causes the longitudinal forward movement of the rack 3 via the pawl 4.

This rear assembly is obtained by the previous putting together, not shown, of the rack 3 and the rear body 2b, by inserting through a rear opening formed in this rear body 2b, until it reaches its position in which its front end 6 is able to engage with the plunger of the cartridge 10. Similarly, there is also another previous step of putting together the lever 1 and the rear body 2b. The lever 1 comprises a horizontal upper part that extends forwards and is intended to be manipulated, and then a part that is inclined downwards and comprises a rear lower end in the form of a pin which is positioned in a corresponding location on the rear body 2b. The pawl 4 is also connected to the lever 1 by a monolithic structure and extends downwards towards the inside of the body 2b. Thus, the rotation pivot of the lever is located towards its rear end and the pawl extends from the inclined part until it makes contact with the teeth of the rack. The horizontal upper part of the lever can thus be actuated downwards in order to move the pawl against the teeth of the rack.

FIG. 2 illustrates an intermediate step in the putting together of the device, in which the device now only comprises two assemblies, front and rear, to be joined together. The front assembly, which is formed by the two put-together front half shells 2a, 2a′ of the body, forms a container holder which surrounds the cartridge 10 and of which only the tool holder 12 and the Luer lock 14 extend beyond the front surface 13 of the container holder. The rear assembly comprises in particular the rear body 2b, the rack 3 and the lever 1 comprising a pawl 4, as was explained above.

FIG. 3 illustrates the device once it has been put together. According to the configuration in FIG. 2, the front and rear assemblies are put together by a step that consists in connecting the front part 6 of the rack 3 to the plunger positioned within the cartridge 10. This connection is produced by screwing, the end 6 of the rack forming a screw that can be screwed into the plunger in the cartridge 10. Alternatively, any other connection can be envisaged.

In addition, this final putting together of the ejecting device comprises fitting the rear body 2b on the container holder. In order for this to be carried out, the rear body 2b has two side tongues 9 that extend longitudinally towards the front and are able to engage with complementary lower and upper rails provided against the side walls inside the container holder 2a, for example between the clips 23a. The end of these tongues 9 has a protuberance 31 that is oriented towards the outside and is intended to engage with corresponding openings 8 in the container holder 2a. Any other equivalent mechanical connection between these two parts could be appropriate. In particular, alternatively, the tongues 9 could be located on the container holder 2a and the corresponding openings 8 in the rear body. In addition, this connection could be of the hook/cleat type, be obtained by a rotational movement of the two parts, by clip-fastening, by a single tongue or hook, etc. The various parts 2a, 2a′ and 2b put together then form the main body of the ejecting device.

At the end of this last step, the ejecting device is ready to use, once a needle, or any other device for guiding the product to be ejected, has been fixed to the tool holder 12, however. Actuating the lever 1 thus ejects the product from the cartridge 10 by the forward movement of the rack 3, which pushes forward the plunger positioned at the rear of the cartridge. For this purpose, the lever 1 is pressed downwards against the container holder 2a by rotating about its rotation pivot which is positioned in its rear part, causing elastic deformation of the pawl 4 resting against the rack 3 about its region of connection to the lever 1, by virtue of the property of flexibility of the material used and the geometry selected in particular at the connection between the pawl 4 and the lever 1. This movement of the pawl 4 enables its toothed end, which engages with the teeth of the rack 3, to exert a pushing force on the rack and to move it forwards within the bore in the body in order to act on the plunger in the cartridge and cause the ejection of the product contained in this cartridge. When the lever is released, the pawl 4 exerts an elastic return force to return it to its initial position while returning the lever 1 to its initial high position. In this phase, a non-return pawl, not shown, formed in the body of the device, prevents the rack 3 from moving backwards again.

FIG. 4 shows a second embodiment of the invention, which differs from the previous embodiment in that the front part or container holder of the body of the ejecting device now only consists of a single part 2a comprising a slot 5 that extends longitudinally over the entire length of the container holder and is wide enough for the syringe or cartridge 10 to pass through until it is positioned in a housing, not shown, formed in the container holder. Alternatively, the part 2a can have a narrow slot and deform elastically in order for the cartridge to pass through. The container holder also comprises mechanical holding means, of the clip type, for example, which are not shown. The cartridge is inserted into its housing in a direction approximately perpendicular to the longitudinal axis x. In the embodiment shown, the slot 5 is located in the top part of the container holder and the cartridge is inserted in a vertical direction z.

However, alternatively, the slot 5 could be located on any other side face of the device, as long as it extends over a sufficient length in the longitudinal direction x to allow the insertion of the cartridge. As a note, it could be possible for the slot 5 not to extend over the entire length of the container holder, as in the embodiment illustrated, but only over a portion extending from the front face 13 of the container holder 2a, such that the cartridge could be inserted in a direction comprising a vertical component z and a longitudinal component x towards the rear in order to push it back, before finalizing its definitive hold in the housing by a movement along a vertical component z. If the slot is not located on the top of the container holder, the same principle remains valid by moving the cartridge closer in a direction perpendicular to the longitudinal direction x at this slot. Next, when the cartridge 10 is positioned in its housing, as is shown in FIG. 5, the rest of the process of putting the device together remains the same as the previous embodiment, and the ejecting device obtained, shown in FIG. 6, which has the particular feature of making it possible to see the cartridge 10 through the slot 5, functions in a similar manner to the device in the first embodiment.

FIGS. 7 to 10 illustrate a variant of the first embodiment, which comprises an additional means for holding the front part of the cartridge, which is subjected to significant forces during the ejection of the product and risks being projected towards the front, as was noted above. Thus, in this variant embodiment, this holding means consists firstly of a groove 11 made in the rear part of the Luer lock 14, and secondly of the ring 21a, 21a′ of the front part 13 of the device, which is housed within this groove 11 while the two half shells 2a, 2a′ are put together, as can be seen in particular in FIG. 10. Thus, the body of the ejecting device holds the front part of the cartridge in position and prevents it from being ejected forwards. Alternatively, the holding means may be in the form of a clip or retaining cleat.

Another variant embodiment of the invention, which is not shown, is obtained by implementing the same solution of holding the cartridge described in the above variant to the second embodiment described above with reference to FIGS. 4 to 6.

According to an advantageous realization of the ejecting device, which relates to all the embodiments described above, the cartridge 10 of the container holder comes as far as the area around the tongues 9 at the openings 8 such that the reverse movement of the rear body 2b, which would require a renewed deformation of the tongues 9 by pressing on the protuberances 31 in order to extract them from the openings 8 while pulling the body 2b towards the rear, is made difficult, if not impossible. This geometry thus obstructs this releasing of the protuberances 31 and makes it possible to lock or virtually lock the body after it has been put together, it being not possible or very difficult to take apart said body, that is to say that putting together is irreversible or virtually irreversible. This realization guarantees the disposable nature of the ejecting device, this being advantageous in order to ensure the safety and hygiene of the device, which can only be used once in order to eject the selected volume of product. This guarantee is thus obtained by the combination of two main factors:
the connection between the two parts 2a, 2b of the body is irreversible or virtually irreversible;
when the plunger is at the end of its travel after ejecting the product, it remains locked in this position by the rack, which is unable to move back on account of the non-return pawl. It is thus not possible to refill the volume of the container holder.

The invention thus has the advantage of making it possible to use a standard rectilinear syringe within a device comprising an ejection controlling mechanism that allows user-friendly ejection of a predefined precise dose without any risk of error.

In order to achieve this advantage, the ejecting device thus comprises a means for introducing a syringe into a housing provided within the front part of its body. This means can either be obtained by a multi-part structure of this front part of the body, it being possible to put these parts together around the body of the syringe, or by a one-part structure comprising a slot extending in a longitudinal direction. In any case, this means makes it possible to introduce the syringe in a direction comprising a non-zero component in a direction perpendicular to the longitudinal axis X. This making it compatible with all the geometries of existing syringes, in particular with those which comprise locking device of the Luer lock type in their front part, said locking device having a greater diameter than the rest of the syringe. Thus, the syringe is introduced by any solution other than longitudinal introduction by way of the rear, which would not enable correct positioning of the voluminous front part of a syringe having a Luer-lock type end piece. Of course, the solution of the invention is also suitable for all syringes, be they standard or non-standard, circular or non-circular in section, with or without end pieces, etc. Thus, it is also suitable for the introduction of a cartridge without a tool holder, the latter being located in a manner directly fixed to the body of the ejecting device.

Thus, embodiments other than those shown can be envisaged. For example, the body of the ejecting device may comprise more than two parts to be put together, these separate parts being obtained by cutting in a longitudinal plane; thus, these parts are put together by bringing them together in a direction approximately perpendicular to the longitudinal axis and thus to the syringe positioned along this axis. According to a second example, the front part of the device comprises a slot which extends longitudinally from its front face over a length at least equal to one third of the length of the syringe 10. In addition, the body of the ejecting device has been shown as being located in at least two parts, front and rear, that are separated along a transverse vertical plane yz. Alternatively, the body of the device could not have this front and rear separation, the part or parts of this body extending in a continuous manner over the entire length of the device.

In addition, the ejecting device has been shown as being similar to the ejecting device described in document FR2913341, by way of example. However, the concept of the invention can be implemented with other types of geometries and with other ejection mechanisms based on levers, buttons, etc.

The invention also relates to the method of manufacturing or putting together an ejecting device, which comprises the essential step of introducing a syringe into a body of the ejecting device in any direction other than longitudinal introduction by way of the rear.

This introduction thus advantageously comprises a direction having a non-zero component in a direction perpendicular to the body, and preferably a direction approximately perpendicular to this body, in particular during its final fixing in its housing within this body.

Alternatively, the cartridge can be introduced by way of the opening in the front face 13 of the body of the ejecting device and no longer by way of a side face. Such a solution would be suitable with a cartridge that does not have a flange in its rear part, unlike the realizations illustrated.

The method of putting together the ejecting device may be based on the putting together of several separate main elements of simple form enabling them to be manufactured from plastic by simple injection moulding. For example, it may consist in manufacturing separately, by injection moulding of plastic, the lever 1, the part or parts 2a, 2a′ forming the container holder, the rear body 2b and the rack 3. The material used may be polyamide, polypropylene, ABS or any other plastic material. A recyclable or biodegradable plastic will be well suited to the device which is disposable after use. A transparent plastic material may also advantageously be used, in particular for the container holder, in order to see the syringe comprising the product to be ejected, at least through a window. Alternatively, the above components may be manufactured differently and from other materials. The rack 3 may for example be made of stainless steel.

Next, the method of manufacturing the ejecting device comprises the following, above-described assembly steps:
putting together a container-holder assembly comprising the cartridge;
putting together a rear assembly comprising a lever, a rear body 2b and a rack 3;
putting together the container-holder assembly and the rear assembly.

The invention also relates to an assembly kit, which comprises the elements essential for assembly, making them easy to transport and allowing optimum hygiene since the final assembly is carried out at the last moment before the ejecting device is used, and ensures that the product packaged in the syringe is preserved in a hygienic manner. This kit could thus comprise the two following elements:
an ejecting device as described above, which is not or partially put together;
a cartridge comprising the product to be ejected.

Of course, the various separate elements of the ejecting device will preferably not have been put together, or possibly partially put together, in this assembly kit, which could thus comprise the following elements: one or more part(s) of a main body, the separate part(s) being separated from one another, a cartridge or syringe containing a product to be ejected, a lever, a rack, and a tool such as a needle.

The device according to the present invention has been designed based on the concept of a disposable device, as was shown above as an advantageous realization. However, non-disposable use, using prior-art cleaning processes, would not depart from the scope of the invention. Specifically, the simplicity of the assembly method makes it just as easy to take apart, for example by modifying the connection between the bodies 2a and 2b in order to aid the releasing of the arms 9. With such a solution, its cleaning would be greatly simplified on account of the simplification of the structure of the device.

In addition, the ejecting device has been described within the scope of a device of the anaesthetic syringe type in the medical field. More generally, the concept implemented is suitable for manufacturing an ejecting device intended for the medial field in order to inject products such as anaesthetics into hard tissues or in order to deposit adhesives, resins or amalgams. It can also be used in the paramedical field, cosmetic surgery, in order to deposit for example predetermined quantities of collagen. It can moreover be used in the field of micromechanics and jewelry in order to produce collages or microwelds or else to deposit any product. Thus, the tool holder 12 present at the end of the cartridge can hold tools other than a needle.

1. Device for ejecting a liquid or pasty product, comprising a body having a housing for holding a cartridge intended to contain the liquid or pasty product to be ejected, wherein the body comprises a means for introducing the cartridge into its housing in a relative direction with respect to the housing, said direction being different from a longitudinal direction by way of the rear.

2. Device for ejecting a liquid or pasty product according to claim 1, wherein the means for introducing the cartridge into its housing makes it possible to introduce the cartridge in a relative direction with respect to the housing, said direction comprising a non-zero component in a plane (yz) perpendicular to the longitudinal axis (x), or in that the means for introducing the cartridge into its housing makes it possible to introduce the cartridge in a relative direction with respect to the housing, said direction being approximately perpendicular to the housing.

3. Device for ejecting a liquid or pasty product according to claim 1, wherein the means for introducing the cartridge into its housing consists of a body of the ejecting device that comprises at least two separate parts that are put together around the cartridge.

4. Device for ejecting a liquid or pasty product according to claim 3, wherein the body of the ejecting device comprises a front container-holder part which is composed of two half shells and a rear part.

5. Device for ejecting a liquid or pasty product according to claim 1, wherein the means for introducing the cartridge into its housing consists of a body of the ejecting device comprising a slot that extends in the longitudinal direction.

6. Device for ejecting a liquid or pasty product according to claim 5, wherein the slot extends over a length that represents at least one third of the length of a front container-holder part of the body of the ejecting device.

7. Device for ejecting a liquid or pasty product according to claim 1, wherein the body of the ejecting device comprises mechanical means for fastening the cartridge, such as clips.

8. Device for ejecting a liquid or pasty product according to claim 1, wherein the body comprises in its rear part a rack that moves translationally and is able to act on a plunger positioned at the rear end of a cartridge intended to contain the product, and a hinged lever and a pawl, for acting on the rack, that form a monolithic structure made of plastic, the lever comprising a cylindrical rear end that forms its rotation pivot.

9. Device for ejecting a liquid or pasty product according to claim 1, wherein it comprises a cartridge arranged in a housing of the body of the device, this cartridge comprising at least one tool holder arranged at least partially in front of the front face of the body of the ejecting device.

10. Device for ejecting a liquid or pasty product according to claim 9, wherein the cartridge comprises a locking device of the Luer lock type for locking the tool to the tool holder, this locking device having a part which has a diameter greater than that of the cartridge arranged in front of the body of the ejecting device, and in that the front face of the ejecting device comprises a circular opening delimited by a ring which has a diameter that corresponds to that of the cartridge.

11. Device for ejecting a liquid or pasty product according to claim 10, wherein the locking device has a groove in which a part of the body of the ejecting device is arranged in order to hold this locking device.

12. Device for ejecting a liquid or pasty product according to claim 9, wherein it comprises a rack that can move translationally and is fixed to a plunger positioned within the cartridge by a screw-type means.

13. Kit for putting together an ejecting device according to claim 1, comprising the following non-assembled separate elements: one or more part(s) of a body comprising a housing and a means for introducing a cartridge into this body, a lever, a rack, and a cartridge comprising a liquid or pasty product to be ejected.

14. Method of putting together a device for ejecting a liquid or pasty product according to claim 1, wherein it comprises a step that consists in introducing a cartridge into the body of the ejecting device in any direction other than longitudinal introduction (x) by way of the rear.

15. Method of putting together a device for ejecting a liquid or pasty product according to claim 14, wherein it comprises a step of putting together at least two separate parts of the body of the device around a cartridge.

16. Method of putting together a device for ejecting a liquid or pasty product according to claim 14, wherein it comprises a step of introducing a cartridge by way of a longitudinal slot in the body of the ejecting device.
